(19) United States
(12) Patent Application Publication
(10) Pub. No.: US 2002/0142466 A1
(43) Pub. Date: Oct. 3, 2002

(54) Methods and compositions for modulating ciliary neurotrophic factor activity

(76) Inventors: Jeffrey S. Flier, West Newton, MA (US); Christian Bjorbaek, Boston, MA (US)

Correspondence Address: Doreen M. Hogle, Esq., HAMILTON, BROOK, SMITH & REYNOLDS, P.C., 530 Virginia Road, P.O. Box 9133, Concord, MA, 01742-9133, US

(21) Appl. No.: 10/040,277

(22) Filed: Oct. 22, 2001

Related U.S. Application Data
(63) Continuation of application No. PCT/US00/10693, filed on Apr. 20, 2000.
(60) Provisional application No. 60/130,172, filed on Apr. 20, 1999.

(51) Int. Cl. A61K 48/00; A61K 38/17; C12N 15/87

(52) U.S. Cl. 435/455; 514/12; 514/44

(57) ABSTRACT

Methods and compositions for altering, or modulating CNTF activity by altering or modulating cytokine inhibitor activity are provided. Specifically encompassed are methods and compositions to alter activity of cytokine inhibitors such as SOCS-1, -2, and -3. SOCS-3 expression is rapidly induced by CNTF treatment in regions of the hypothalamus that are known to be involved in the regulation of body weight. As described herein, a SOCS-3-mediated CNTF cell-signaling inhibitory pathway exists, suggesting that SOCS-3 is a negative regulator of CNTF signal-transduction in the brain. Since CNTF treatment of animals suppresses appetite and induces weight loss, inhibition of SOCS-3 expression or activity is a potential target for the development of drugs aimed at improving CNTF sensitivity or prolonging CNTF activity in a mammal and inducing weight loss. Thus, altering SOCS-3 activity provides a means for modulating CNTF-induced cell signaling and therefore modulating bodyweight.

### RELATED APPLICATION(S)

This application is a continuation of PCT/US00/10693 filed Apr. 20, 2000 which was published in English and which claims the benefit of the Provisional Application No. 60/130,172 filed on Apr. 20, 1999, the entire teachings of which are incorporated herein by reference.

### GOVERNMENT SUPPORT

The invention was supported, in whole or in part, by grant DK R37 28082 from the National Institutes of Health to J. S. Flier. The Government has certain rights in the invention.

### BACKGROUND

Ciliary neurotrophic factor (CNTF) is a neurocytokine normally expressed in glial cells that acts on both neural and non-neural cells to promote gene expression, survival and differentiation (Segal, R. A., and Greenberg, M. E., Annu. Rev. Neurosci., 19:463-489 (1996); Ip, N. Y., and Yancopoulos G. D., Annu. Rev. Neurosci., 19:491-515 (1996)). CNTF was initially identified as a trophic factor which induced survival of embryonic ciliary ganglion neurons (Adler, R., et al., Science, 204:1434-1436 (1979)), but was later shown to belong to the superfamily of cytokines which includes interleukin-6 (IL-6), leukemia inhibitory factor (LIF) and leptin (Lin, L. F., et al., Science, 246:1023-1025 (1989); Stockli, K. A., et al., Nature, 342:920-923 (1989); Bazan, J. F., Neuron, 7:197-208 (1991); Zhang, F., Nature 387:206-209 (1997)). The expression of CNTF mRNA has been shown to increase in the region of a mechanically lesioned site in the nervous system (Ip, N. Y., et al., Eur J Neurosci., 5:25-33 (1993)). This evidence suggests that CNTF may play an important role in the response to neuronal injury (Ip, N. Y., and Yancopoulos G. D., Annu. Rev. Neurosci., 19:491-515 (1996)).

CNTF activates intracellular signal transduction by binding to a membrane-bound heterotrimeric receptor complex consisting of a ligand-specific subunit (CNTFRα) (Davis, S., et al., Science, 253:59-63 (1991)), the leukemia inhibitory factor receptor subunit, and gp130 (Davis, S., et al., Science, 260:1805-1808 (1993)). The CNTFα receptor subunit is expressed throughout the brain (Ip, N. Y., et al., Neuron, 10:89-102 (1993); Kordower, J. H., et al., J. Comp. Neurol., 377:365-380 (1997)), and specific localization of CNTFRα has been demonstrated in the arcuate nucleus and other parts of the hypothalamus (Gloaguen, I., Proc. Natl. Acad. Sci., USA, 94:6456-6461 (1997)). In rodents, it has been noted that peripheral administration of CNTF results in fever and transient reduction in food intake (Espat, N. J., et al., Am. J. Physiol., 271:R185-R190 (1996); Shapiro, L, et al., Proc. Natl. Acad. Sci. USA, 90:8614-8618 (1994); Henderson, J. T., et al., J. Clin. Invest., 93:2632-2638 (1994); Martin, D., Am. J. Physiol., 271:R1422-R1428 (1996)). Recent data also show that peripherally administered CNTF reduces the body weight of ob/ob and db/db mice (Plata-Salaman, C. R., Nutrition, 11:674-677 (1995)).

Cytokine stimulation induces members of the STAT transcription factor family to “dock” onto receptor phosphotyrosines, enabling their own tyrosine phosphorylation by JAK tyrosine kinase family members (Heinrich, P. C., et al., Biochem., J., 334:297-314 (1998)). Subsequently, STAT proteins translocate to the nucleus and bind to conserved genomic regulatory sequences to provide a rapid means of activating gene transcription (Heinrich, P. C., et al., Biochem., J., 334:297-314 (1998)).

Recently, a new family of cytokine-inducible inhibitors of signaling has been identified including CIS (cytokine-inducible sequence), SOCS-1 (suppressor of cytokine signaling), SOCS-2 and SOCS-3 (Starr R et al, Nature 387:917-921, 1997; Endo T A, et al, Nature 387:921-924, 1997; Naka T et al, Nature, 387:924-929, 1997; Masuhara M et al, Biochem Biophys Res Commun, 239:439-446, 1997). IL-6, LIF, growth hormone (GH) and erythropoietin (EPO) induce transcriptional activation of one or more of the CIS or SOCS genes in vivo and in vitro, through activation of the JAK-STAT pathway (Starr, 1997; Endo, 1997; Naka, 1997; Yoshimura et al. Embo J. 14:2816-2826, 1995, Masuhara, 1997).

### SUMMARY OF THE INVENTION

As described herein, CNTF strongly induces SOCS-3 mRNA in the arcuate nucleus of the hypothalamus, a region known to express SOCS-3 mRNA after leptin treatment and to be a key target of leptin action. These data are consistent with the role of arcuate nucleus as a key site for the regulation of body weight by CNTF. As further demonstrated herein, SOCS-3 is an inhibitor of CNTF signal transduction.

The present invention encompasses methods and compositions for altering, or modulating CNTF activity by altering or modulating cytokine inhibitor activity. Specifically encompassed in the present invention are methods and compositions to alter activity of cytokine inhibitors such as SOCS-1, -2, and -3. As demonstrated herein, SOCS-3 expression is rapidly induced by CNTF treatment in regions of the hypothalamus that are known to be involved in the regulation of body weight. It has now been determined that a SOCS-3-mediated CNTF cell-signaling inhibitory pathway exists, suggesting that SOCS-3 is a negative regulator of CNTF signal-transduction in the brain. As described herein, it is reasonable to expect that SOCS-3 activity is an important factor in attenuation of CNTF signaling. Since CNTF treatment of animals suppresses appetite and induces weight loss, inhibition of SOCS-3 expression or activity is a potential target for the development of drugs aimed at improving CNTF sensitivity or prolonging CNTF activity in a mammal and inducing weight loss. Thus, as described herein, altering SOCS-3 activity provides a means for modulating CNTF-induced cell signaling and therefore modulating bodyweight.

Mechanical lesion of the animal nervous system results in increased CNTF production at the lesioned site. Therefore, the present invention also relates to methods of treating central nervous system injury in mammals by inhibiting SOCS-3 activity. The inhibitors of the present invention (such as small molecule drugs)

which inhibit the activity of SOCS-3 may be administered to prolong the cell-survival effects of endogenous produced CNTF or administered CNTF. Furthermore, CNTF induces cell survival after retinal degeneration and is known to prevent cell death in neuronal cell cultures expressing Huntington protein. Therefore, SOCS-3 inhibitors of the present invention prolong the cell-survival effects of endogenous or administered CNTF for treatment of neurodegenerative disorders like Huntington's disease and retinal injury.

The present invention therefore pertains to methods of modulating CNTF cell signaling by altering SOCS-3 activity in a mammal to modulate body weight, modulate glucose level in the blood and promote healing of CNS or retinal injury. Furthermore, the present invention pertains to a method of preventing neuronal cell death, e.g. in response to Huntington protein and to prevent gut epithelial cell loss.

The present invention is drawn to a method of modulating ciliary neurotrophic factor cell signaling activity in a cell, comprising contacting said cells with an modulator of SOCS-3 activity.

The present invention is drawn to cell lines comprising a cytokine receptor and a reporter gene construct, wherein contacting said cytokine receptor with its cognate ligand results in SOCS-3 production, and wherein transcription of the reporter gene is inhibited by SOCS-3.

The present invention is drawn to a method for identifying inhibitors of SOCS-3 activity, comprising the steps of: contacting the cells of the present invention with an organic molecule library comprising candidate SOCS-3 inhibitors or transfecting said cells with a cDNA expression library comprising DNA encoding candidate SOCS-3 inhibitors. The cells are then contacted with ciliary neurotrophic factor and selected for those having increased reporter gene activity. The organic molecule or cDNA that resulted in increased reporter gene activity are identified.

The present invention is drawn to a ciliary neurotrophic factor responsive cell line, wherein the cell line is dependent upon a second cytokine (e.g., IL-3) for growth.

The present invention is drawn to a method for identifying inhibitors of SOCS-3 activity comprising the steps of: culturing CNTF dependent cells that are capable of growing in the presence of IL-3 under conditions suitable for growth. The cells are then removed from the presence of IL-3 and contacted with an organic molecule library comprising candidate SOCS-3 inhibitors or transfecting with a cDNA expression library comprising candidate SOCS-3 inhibitors and with ciliary neurotrophic factor. Cells capable of proliferating in the presence of ciliary neurotrophic factor are then selected. The organic molecule or cDNA from the selected cells is identified.

The present invention is drawn to SOCS-3 inhibitors identified as described herein.

The present invention is drawn to a method of reducing weight or food intake in a mammal, comprising administering an effective amount of a SOCS-3 inhibitor to said mammal. The present invention is also drawn the manufacture of a medicament comprising a SOCS-3 inhibitor, for the reduction of weight or food intake in a mammal.

The present invention is drawn to a method of reducing weight or food intake in a mammal, comprising administering an effective amount of ciliary-neurotrophic factor in combination with a SOCS-3 inhibitor to said mammal. The present invention is also drawn the manufacture of a medicament comprising ciliary neuotrophic factor and a SOCS-3 inhibitor, for the reduction of weight or food intake in a mammal.

The present invention is drawn to a method of preventing or inhibiting neurodegeneration in a mammal, comprising administering an effective amount of a SOCS-3 inhibitor to said mammal. The present invention is also drawn the manufacture of a medicament comprising a SOCS-3 inhibitor, for the prevention or inhibition of neurodegeneration in a mammal.

A method of preventing or inhibiting neurodegeneration in a mammal, comprising administering an effective amount of ciliary neurotrophic factor in combination with a SOCS-3 inhibitor to said mammal. The present invention is also drawn the manufacture of a medicament comprising ciliary neurotrophic factor and a SOCS-3 inhibitor, for the prevention or inhibition of neurodegeneration in a mammal.

The present invention is drawn to a method of increasing weight or food intake in a mammal, comprising administering an effective amount of ciliary neurotrophic factor inhibitor to said mammal. The present invention is also drawn the manufacture of a medicament comprising a ciliary neurotrophic factor inhibitor, for increasing of weight or food intake in a mammal.

Thus, as a result of the present invention methods and compositions are now available to modulate CNTF activity, specifically by modulating the activity of the cytokine inhibitors, SOCS-3 or SOCS-1.

### BRIEF DESCRIPTION OF THE DRAWINGS

FIG. 1A is an autoradiogram of ³²P-labeled RT-PCR products of CIS, SOCS-1, SOCS-2 and SOCS-3 mRNA.

FIG. 1B is a Northern blot of SOCS-3 mRNA in murine hypothalami after CNTF treatment.

FIG. 2A is a dark field photo micrograph of an emulsion-dipped slide of rat hypothalamic sections hybridized with SOCS-3 antisense ³⁵S-labeled RNA probes after treatment of the animal with pyrogen-free saline.

FIG. 2B is a dark field photo micrograph of an emulsion-dipped slide of rat hypothalamic sections hybridized with SOCS-3 antisense ³⁵S-labeled RNA probes after treatment of the animal with CNTF.

FIG. 2C is a dark field photo micrograph of an emulsion-dipped slide of rat hypothalamic sections hybridized with SOCS-3 antisense ³⁵S-labeled RNA probes after treatment of the animal with leptin.

FIG. 3A is an autoradiograph of an STAT1 and STAT3 electrophoretic mobility shift assay after CNTF treatment of astrocytes.

FIG. 3B shows the results of a Northern blot of SOCS-3 mRNA induced by CNTF treatment of astrocyte cells.

FIG. 3C shows the results of a Western blot of SOCS-3 protein induced by CNTF treatment of astrocyte cells.

FIG. 4 is a bar graph showing SOCS-3 mediated inhibition of CNTF induced gene expression.

FIG. 5A is an autoradiogram of ³²P-labeled RT-PCR products of CIS, SOCS-1, 2 and 3, isolated from cerebellum after the indicated treatment.

FIG. 5B is an autoradiogram of ³²P-labeled RT-PCR products of CIS, SOCS-1, 2 and 3, isolated from kidney after the indicated treatment.

FIG. 5C is an autoradiogram of ³²P-labeled RT-PCR products of CIS, SOCS-1, 2 and 3, isolated from liver after the indicated treatment.

### DETAILED DESCRIPTION OF THE INVENTION

The present invention pertains to regulation of CNTF activity in the brain and other tissues such as kidney and liver. The present invention pertains to regulation of the CNTF-induced cell signaling pathway in the hypothalamus via regulation of cytokine inhibitor activity in particular, SOCS-1 and SOCS-3 activity.

Recently, a new family of cytokine inhibitors has been described, including CIS and SOCS-1, 2, and 3. CIS, an inhibitor of cytokine receptor signaling, is thought to bind directly to specific cytokine receptors and possibly block accessibility of signaling molecules to key phosphotyrosine residues of the receptor (Yoshimura, et al., EMBO J. 14:2816-2826 (1995)). SOCS-1, 2 and 3 are cytokine inducible inhibitors that were found to be active in hematopoietic cells (Starr, et al., Nature 387:917-921, 1997; Endo et al., Nature 387:904-929 (1997); and Naka, et al., Nature 387:924-929 (1997).

As demonstrated herein, peripheral administration of recombinant CNTF to normal rats and ob/ob mice induced SOCS-3 mRNA in specific areas of the hypothalamus. In addition to the circumventricular organs and the ependymal lining of the ventricles, in situ hybridization of brain sections from CNTF treated rats revealed significant SOCS-3 mRNA expression in the arcuate nucleus of the hypothalamus. The region of the arcuate nucleus expressing SOCS-3 mRNA

overlaps with that expressing several neuropeptides involved in regulation of body weight. As demonstrated herein, CNTF specifically induces expression of SOCS-3 mRNA in hypothalamic nuclei known to express neuropeptides involved in regulation of body weight including neuropeptide Y (NPY), pro-opiomelanocortin (POMC), agouti-related protein (AGRP) and cocaine-and amphetamine-regulated transcript (CART) (Ollmann, M. M., et al., Science, 278:135-138 (1997); Kristensen, P., et al., Nature, 393:72-76 (1998); Stanley, B. G. and Leibowitz, S. F., Proc. Natl. Acad. Sci. USA 82:3940-3943 (1985); Mountjoy, K. G., and Wong, J., Mol. Cell. Endocrinol., 128:171-177 (1997)). In addition, SOCS-3 mRNA is also induced in the arcuate nucleus by leptin (Bjørbaek C, et al., Molecular Cell 1:619-625 (1998)), which is known to regulate expression of each of these neuropeptides in vivo (Stephens, T. W., et al., Nature, 377:530-532 (1995); Schwartz, M. W., et al., Diabetes, 45:531-535 (1996); Thornton, J. E., et al., Endocrinology, 138:5063-5066 (1997); Cheung, C. C., et al., Endocrinology, 138:4489-4492 (1997); Ollmann, M. M., et al., Science 278:135-138 (1997); Kristensen, P., et al., Nature 393:72-76 (1998)). Anorexia has been noted after peripheral injection of CNTF into animals and humans (Gloaguen, I., Proc Natl Acad Sci USA, 94:6456-6461 (1997);Henderson, J. T., et al., J. Clin. Invest., 93:2632-2638 (1994); Miller, R. G., et al., Ann. Neurol., 39:256-260 (1996)). The results described herein are consistent with the possibility that the CNTF induced anorexia reasonably involves regulation of one of more of these neuropeptides and that modulation of CNTF activity can be affected by modulation of SOCS activity.

In the brain, dense SOCS-3 hybridization signals were detected in the median eminence and the subfornical organ after peripheral CNTF administration. In addition, robust signals were seen in the ependymal lining of all ventricles. Therefore, as demonstrated herein, CNTF may reach the cerebrospinal fluid, possibly via diffusion through the fenestrated vessels in the circumventricular organs. Cells within the circumventricular organs are clearly also highly responsive to peripheral administration of CNTF. Once in the cerebrospinal fluid, CNTF can induce SOCS-3 mRNA expression by activation of CNTF receptors expressed on the ependymal cells which comprise the lining of the ventricles. CNTF can also bind to soluble CNTFRα proteins, which have been reported to be present at this site (Davis, S., et al. Science, 259:1736-1739 (1993)). In addition, in response to injury in the CNS, ependymal cell proliferation increases dramatically. The soluble CNTF-CNTFRα complex may act on cells that normally do not respond to CNTF (Ip, N. Y. and Yancopoulus, G. D., Annu. Rev. Neurosci. 19:491-515 (1996)).

As demonstrated herein, a significant number of the cells activated by CNTF in the arcuate nucleus are neurons, a result which is consistent with data demonstrating expression of CNTFRα in neurons (Ip, N. Y., et al., Neuron, 10:89-102 (1993); Kordower, J. H., et al., J. Comp. Neurol., 377:365-380 (1997)). However, as demonstrated by results using the astrocyte cell line, some SOCS-3 positive cells in this region may be of glial origin. In addition, these results are also consistent with the possibility that non-neuronal cells comprising the ependymal lining of the ventricles and glial populations within the circumventricular organs are direct targets of peripherally administered CNTF.

Ligand binding to CNTF receptors leads to activation of receptor-bound JAK kinases, which phosphorylate tyrosines in the cytoplasmic domain of the receptor as well as in other cytoplasmic target proteins. Several pathways can be activated by JAK kinases, including the signal transducers and activators of transcription (STAT), ras/mitogen-activated protein kinase, and phospho-inositide-3 kinase pathways. As shown in Example 1, CNTF stimulation in vitro results in the induction of SOCS-3 mRNA. Furthermore, as shown in Example 1, when SOCS-3 is present, CNTF induced signaling by a STAT-reporter construct is inhibited.

Based on the results described herein, it is reasonable to expect that in the hypothalamus in general, and arcuate nucleus in particular, SOCS-3 antagonizes CNTF induced cell-signaling by interacting with JAK2 and competing with binding between JAK2 and its substrate (CNTF receptor or STAT). As demonstrated herein, SOCS-3-mediated CNTF cell-signaling inhibitory pathway exists in the hypothalamus. SOCS-3 therefore, can negatively regulate cell signaling via the CNTF receptor.

As defined herein, modulating (also referred to herein as altering, adjusting or regulating) CNTF activity means inhibiting or enhancing the biological activity of CNTF by inhibiting or activating cytokine inhibitors such as SOCS-3. Inhibiting CNTF activity encompasses partial inhibition as well as complete abrogation of CNTF activity. Inhibiting SOCS-3 activity encompasses partial inhibition as well as complete abrogation of CNTF activity.

The biological activity of CNTF is defined herein as the ability of CNTF to activate one or more signal transduction pathways in a cell as a result of interaction between (e.g., binding) CNTF and a CNTF receptor associated with the cell. CNTF activity can be measured as the level of phosphorylation of the receptor, JAK2 or STAT3 CNTF activity can be measured as the amount of JAK2 or STAT3 activity. Further, CNTF activity can be measured by the amount of gene transcription from STAT3 responsive genes (STAT3 activity).

As defined herein, SOCS activity and in particular, SOCS-3, SOCS-1 or SOCS-2 mediated CNTF cell signaling, is the inhibition or inactivation (completely or partially) of CNTF induced cell signaling. As demonstrated by the present invention, SOCS protein and in particular SOCS-3 and SOCS-1 mediate the down regulation of CNTF signaling as measured by lack of phosphorylation of CNTF receptor, JAK2 or STAT3, as well as by the association of JAK2 and said SOCS protein. As described herein, transcription of SOCS gene(s) such as SOCS-3 and SOCS-1 is part of a negative feedback loop triggered by CNTF activation of the CNTF receptor. CNTF may also participate in a negative feedback loop wherein CNTF activation of the CNTF receptor induces a cytokine inhibitor molecule such as SOCS-3 or SOCS-1, which in turn binds to and inhibits signaling through a separate cytokine receptor such as the leptin receptor.

The present invention encompasses methods and compositions for modulating CNTF activity comprising altering SOCS-3 activity in a cell. In one embodiment of the present invention, CNTF activity is upregulated or increased via inhibition of SOCS-3 or SOCS-1 activity.

SOCS activity can be inhibited by inhibiting or reducing the amount of SOCS protein expressed in a cell, or by introducing a polynucleotide encoding a modified SOCS protein into a cell, wherein the modified SOCS protein includes a mutant, variant, derivative, or analog of the SOCS protein.

SOCS protein activity can also be inhibited by transfecting the cell with a polynucleotide construct, wherein the construct encodes an altered, or modified polypeptide such as altered or modified SOCS-3 or SOCS-1 protein, polypeptide or peptide. In one embodiment, the modified SOCS-3 or SOCS-1 polypeptide is a competitive inhibitor (e.g., antagonist) of endogenous SOCS-3 or SOCS-1. The modified protein, polypeptide or peptide can interact with a SOCS-3 or SOCS-1 target protein (e.g., JAK2), without interfering with the activity of the target protein. SOCS-3 or SOCS-1 activity can also be inhibited or reduced by introducing a SOCS-3 or SOCS-1 inhibitor into the cell. Such an inhibitor can be a peptide or small organic molecule that interferes with SOCS-3 or SOCS-1 activity. Such an inhibitor can interact specifically with SOCS-3 or SOCS-1, or to its intended target, to inhibit SOCS-3 or SOCS-1 activity. For example, the inhibitor can interact with downstream targets of SOCS-3 or SOCS-1 such as JAK2.

In one embodiment, cells of interest are contacted with a SOCS-3 or SOCS-1 inhibitor, resulting in increased CNTF activity in the cell. Increasing CNTF activity results in the increase of the CNTF cell-signaling pathway, resulting in, inter alia, weight loss, lowered blood glucose levels, increase in body temperature and enhancement of repair of injured brain tissue. Thus, a polypeptide or peptide inhibitor/antagonist comprising the SOCS-3 amino acid sequence of GenBank Accession Number U88328, or a modified SOCS-3 amino acid sequence, or an active fragment thereof, can interact with SOCS-3 and/or its target, e.g., JAK2, resulting in diminished ability of SOCS-3 to inhibit CNTF activity.

An increase in CNTF activity includes CNTF activity being prolonged and CNTF activity being enhanced, whereby less CNTF produces the same effect.

The present invention further encompasses methods of increasing or enhancing SOCS-3 or SOCS-1 activity in a cell. Increased SOCS-3 or SOCS-1 activity in a cell can inhibit or reduce CNTF-induced cell signaling. A reduction or inhibition of CNTF-induced cell signaling can be useful to promote weight gain in an individual. SOCS-3 or SOCS-1 activity can be increased by transfecting a cell with a polynucleotide construct encoding a biologically active form of SOCS-3 or SOCS-1 protein, or a biologically active fragment thereof. In another embodiment, SOCS-3 or SOCS-1 activity can be increased by transfecting a cell with a nucleic acid encoding a modified SOCS-3 or SOCS-1 protein that has increased biological activity. In still another embodiment, cells of interest can be treated with a substance such that endogenous SOCS production is increased. Such treatment can result in a transient increase or a chronic increase in SOCS production.

The present invention also pertains to methods of evaluating SOCS-3 or SOCS-1 mediated CNTF activity and methods of screening candidate SOCS-3 or SOCS-1 inhibitors, antagonists and agonists of activity. Inhibitors of SOCS-3 or SOCS-1 activity can be identified and tested in in vitro assays and in ex vivo cell-based assays, as described herein. Candidates exhibiting the desired activity in vitro or ex vivo can be further evaluated in art-accepted animal models.

Candidate antagonists/agonists can be assessed for their ability to inhibit/enhance SOCS-3 or SOCS-1 activity, by their ability to allow reporter gene expression or cell proliferation of SOCS-3 or SOCS-1 expressing cells comprising the steps of: culturing the cells described above under conditions suitable for maintenance and growth; contacting said cells with the candidate molecule or an organic molecule library comprising SOCS-3 or SOCS-1 inhibitors or transfecting the cells with a cDNA expressing the candidate molecule with a cDNA expression library comprising DNA encoding candidate SOCS-3 or SOCS-1 inhibitors; contacting the cells with CNTF; selecting the cells having increased reporter gene activity and identifying the organic molecule or cDNA that had contacted the selected cells. Methods of measuring gene transcription and the enhancement or inhibition thereof are well known to those of skill in the art.

For example, a cell line that is capable of expressing SOCS-3 or SOCS-1 in response to treatment with the appropriate cytokine, and also expresses a cytokine receptor and contains a reporter gene construct is treated with at least one candidate

inhibitor. The reporter gene construct is designed such that transcription of the reporter gene construct is inhibited by the presence of SOCS-3 or SOCS-1 protein. After exposure of the cells to the candidate inhibitor, the cells are screened for increased expression of the reporter gene in comparison to untreated cells. Inhibitors that result in increased expression are identified and/or isolated. In one embodiment, the cytokine receptor is the CNTF receptor. In one embodiment, the cell line expresses the leukemia inhibitory factor receptor (LIFR) together with gp130 and ciliary neurotrophic binding protein (CNTFα). In another embodiment, the cell line expresses LIFR and gp130 and is provided with soluble CNTFα and CNTF. In one embodiment, the reporter gene construct is a CNTF responsive promoter attached to a reporter gene. CNTF responsive promoters are well known in the art. The reporter gene can be the CAT gene, the luciferase gene or the β-galactosidase gene.

The present invention further encompasses a cytokine dependent cell line wherein the cells stably express SOCS-3 or SOCS-1 and the CNTF receptor. For example, the cytokine can be IL-3, or other related cytokines. In one embodiment, the cytokine dependent cell line is Ba/F3 cells which express CNTF receptors together with SOCS-1 or SOCS-3. The CNTF receptor can be expressed transiently or stably. In another embodiment, the cytokine dependent cell line expresses leukemia inhibitory factor receptor (LIFR) and gp130, such that exposure to soluble CNTFα and CNTF results in CNTF signal transduction. The invention further provides a method of isolating and identifying inhibitors of SOCS-3 or SOCS-1, comprising the steps of culturing the cytokine-dependent cells described above in the presence of said cytokine under conditions suitable for maintenance and growth; removing said cells from the cytokine (in the case of BA/F3, the cytokine would be IL-3), contacting the cells with a candidate organic molecule or with a library comprising SOCS-3 or SOCS-1 inhibitor molecules or transfecting said cells with a cDNA expressing a SOCS-3 or SOCS-1 candidate inhibitor or a cDNA expression library comprising DNA encoding candidate SOCS-3 or SOCS-1 inhibitors; selecting cells capable of proliferating in the presence of CNTF (or soluble CNTFα

in embodiments where cells express LIFR and gp130) and identifying the organic molecule of cDNA that contacted the cells selected as described. Methods to transfect cells with cDNA expression libraries and subsequently isolate the cDNA are well known in the art.

The present invention also pertains to the cell lines used to evaluate SOCS-3 or SOCS-1 mediated CNTF activity and used to screen candidate SOCS-3 or SOCS-1 inhibitors.

The cell lines of the present invention can be used to screen libraries such as peptide libraries, organic molecule libraries or cDNA libraries to select and identify molecules that inhibit (or enhance) SOCS-3 or SOCS-1 activity. In one embodiment, treatment of cells comprises contacting the cells with an organic molecule library. In another embodiment, the cells are contacted with a peptide library. In still another embodiment, the cells are transfected with a cDNA expression library.

The present invention includes cell lines suitable for use in the screening methods described herein. Any suitable cell type can be used in the screening methods. In one embodiment, the cells are hypothalamic cells or ependymal cells. In another embodiment, the cells are neural cells, kidney cells, liver cells, or blood. Cells encompassed by the present invention can be found in all vertebrates including mammals and humans. In another, embodiment, the cells are maintained in a cell line, e.g., transformed cells, which are suitable, for example, for use in testing CNTF, SOCS-1, -2 or -3 activity. In one embodiment, the cell line is a mammalian cell line such as CHO cells, Ba/F3 cells, HepG2 cells or H35-hepatoma cells, wherein said cells stably express a cytokine receptor and a reporter gene construct wherein the reporter gene construct is active in the absence of SOCS-3 or SOCS-1. The cell line is further modified by the introduction of SOCS-3 or SOCS-1 whereby the reporter gene construct is inhibited by SOCS-3 expression respectively. In one embodiment, the cytokine receptor is the CNTF receptor. In another embodiment, the reporter gene encodes luciferase. In another embodiment, the reporter gene encodes β-galactosidase. In a further embodiment, the reporter gene construct contains SOCS-3 or SOCS-1 promoter elements. In a preferred embodiment, the cell lines, cell signaling components (such as CNTF receptors, JAK2), SOCS-3 or SOCS-1 are of human origin.

Peptides or small organic molecules can be evaluated for their ability to specifically interact with SOCS-3 or SOCS-1 in standard binding or capture assays known in the art. For example, SOCS-3 or SOCS-1 can be immobilized to a suitable surface (such as wells of a plastic microtiter plate or on beads) and contacted under physiological conditions to the peptide library or organic molecule library. Peptides or organic molecules that bind SOCS protein are used in cell-based assays to measure their effect on SOCS activity. In one embodiment, the peptide or organic molecules are labeled for subsequent detection. In another embodiment, the peptide or small organic molecule library; the antibody or antibody fragments or the target molecule or target molecule derivatives can be immobilized on a solid support and contacted with SOCS-3 or SOCS-1.

In one embodiment, peptide libraries, such as an oriented peptide library (Z. Songyang et al. Cell 72:767, 1993; are screened for peptides that interact with SOCS-3 or SOCS-1. Peptide libraries and other small organic molecule libraries can also be screened using other assays known in the art, such as proximity assays or Biospecific Interaction Analysis (BIA). Biospecific Interaction Analysis (BIA) in real time can be performed to evaluate candidate molecules for their ability to bind SOCS-3 or SOCS-1. Surface plasmon resonance (SPR), which is the basis for BIA measurements, is an optical phenomenon arising in metal films under conditions of total internal reflection. The phenomenon produces a sharp dip in the intensity of reflected light at a specific angle. The position of this resonance angle depends on several factors, including the refractive index of the medium close to the non-illuminated side of the metal film. Refractive index is directly related to the concentration of dissolved material in the medium. By keeping other factors constant, SPR is used to measure changes in the concentration of macromolecules in a surface layer of solution in contact with a dextran-coated gold film. Using the BIAcore™ instrument from Pharmacia Biosensor AB, the association and dissociation rate constants for a peptide or organic molecule binding to SOCS-3 or SOCS-1 can be measured. Polypeptides peptides, peptide mimics or small organic molecules exhibiting higher association constants (Kₐ) have the greatest potential for ability to interact with SOCS-3 or SOCS-1 and inhibit their activity. Peptides or organic molecules with or without prior screening for ability to bind SOCS protein can be tested in cell lines for effect on CNTF activity.

Candidate inhibitors/agonists can further be evaluated in animal models. Animal models where SOCS-3 or SOCS-1 activity can be evaluated are known in the art, for example see Leibel et al., J. Biol. Chem. 272:319337-319340, 1997. Inhibitors identified as described by the present invention can be useful to treat obesity or prevent weight gain in a mammal. Such molecules can also be useful to treat brain injury.

The present invention is useful to study the role of CNTF and SOCS activity in weight gain and or/loss and in the prevention of neurodegeneration. The present invention encompasses methods of reducing food intake in a mammal comprising increasing CNTF cell-signaling comprising inhibiting SOCS-3 or SOCS-1 activity. In one embodiment, the mammal loses body weight in response to treatment with the inhibitors of the present invention. In another embodiment, the inhibitors of the present invention enhance or prolong the effect of endogenous or administered CNTF to prevent or minimize neuronal damage, obesity, impotency and to maintain gut epithelial cells.

As defined herein, modified SOCS-3 or SOCS-1 encompasses SOCS-3 or SOCS-1 molecules comprising fragments, derivatives, analogs, variants and mutants there. These modified SOCS-3 or SOCS-1 molecules possess SOCS-3 or SOCS-1 inhibitor/antagonist activity, thereby inhibiting the activity of endogenous SOCS-3 or SOCS-1 present in a cell, resulting in an increase of CNTF activity.

Another activity of modified SOCS-3 or SOCS-1 molecules is the antigenic property of the modified SOCS-3 or SOCS-1 molecule comprising the ability of the modified SOCS-3 or SOCS-1 or SOCS-1 to bind to SOCS-3 or SOCS-1 specific antibodies. The modified SOCS-3 or SOCS-1 molecule can also possess immunogenic properties whereby the modified SOCS-3 or SOCS-1 molecule induces an immunogenic response, e.g., the production of antibodies that specifically bind to endogenous (native) SOCS-3 or SOCS-1 respectively.

A fragment of SOCS-3 or SOCS-1 encompasses polypeptides that comprise only a part of the full-length protein and inhibit endogenous SOCS-3 or SOCS-1 activity. Such fragments can be produced by amino and/or carboxyl terminal deletions, as well as internal deletions. Fragments can also be produced by enzymatic digestion. Such modified molecules can be tested for inhibitory activity as described herein.

“Derivatives” and “variants” of SOCS-3 or SOCS-1 include truncated and hybrid forms of the protein. “Truncated” forms are shortened forms, for example with deletions at one or both termini of the protein and/or internal deletions of the protein. “Hybrid” forms of SOCS molecules comprise a portion of the SOCS amino acid sequence from another protein, e.g., a different SOCS protein, or a protein unrelated to SOCS, for example.

“Variants” and “mutants” of SOCS-3 or SOCS-1 can be produced using in vitro and in vivo techniques well-known to those of skill in the art, for example, site-specific mutagenesis and oligonucleotide mutagenesis. Manipulations of the SOCS-3 or SOCS-1 protein sequence can be made at the protein level as well. Any of a number of chemical modifications can be carried out by known techniques including, but not limited to, specific chemical cleavage by cyanogen bromide, trypsin and papain. SOCS-3 or SOCS-1 can also be structurally modified or denatured, for example, by heat. In general, mutations can be conservative or non-conservative amino acid substitutions, amino acid insertions or amino acid deletions. The mutations can be at or near SOCS-3 or SOCS-1 binding or active sites, such sites are involved, for example, in SOCS-3 or SOCS-1 mediated inhibition of cell-signaling.

In one embodiment, DNA encoding a SOCS-3 or SOCS-1 mutant is prepared by site-directed mutagenesis of DNA that encodes the SOCS-3 or SOCS-1. Site-directed (site-specific) mutagenesis allows the production of SOCS-3 variants through the use of specific oligonucleotide sequences that encode the DNA sequence having the desired mutation, as well as a sufficient number of adjacent nucleotides, to provide a primer sequence of sufficient size and sequence complexity to form a stable duplex on both sides of the deletion junction being traversed. Typically, a primer of about 20 to 25 nucleotides in length is preferred, with about 5 to 10 residues on both sides of the junction of the sequence being altered. In general, the techniques of site-specific mutagenesis are well known in the art. Alternatively, nucleotide substitutions can be introduced by synthesizing the appropriate DNA fragment in vitro, and amplifying it by PCR procedures known in the art.

In general, directed mutagenesis can be performed by first obtaining a single-stranded vector that includes within its sequence a DNA sequence that encodes the relevant protein. An oligonucleotide primer bearing the desired mutated sequence is prepared, generally synthetically, for example, by the method of Crea et al., Proc Natl Acad Sci USA. 75:5765, 1978. This primer can then be annealed with the single-stranded protein sequence-containing vector, and subjected to DNA polymerizing enzymes such as E. coli polymerase I Klenow fragment, to complete the synthesis of the mutation-bearing strand. Thus, a heteroduplex is formed wherein one strand encodes the original non-mutated sequence and the second strand bears the desired mutation. This heteroduplex vector can then be used to transform appropriate host cells such as JM 101 cells, and clones can be selected that include recombinant vectors bearing the mutated sequence. Thereafter, the mutated region can be removed, if necessary, and placed in an appropriate expression vector for protein production.

The PCR technique can also be used in creating amino acid sequence variants of SOCS-3 or SOCS-1. When small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template. For introduction of a mutation into a plasmid DNA, one of the primers can be designed to overlap the position of the mutation and to contain the mutation; the sequence of the other primer is preferably identical to a stretch of sequence of the opposite strand of the plasmid, but this sequence can be located anywhere along the plasmid DNA. It is preferred, however, that the sequence of the second primer is located within 500 nucleotides from that of the first, such that in the end the entire amplified region of DNA bounded by the primers can be easily sequenced. PCR amplification using a primer pair like the one just described results in a population of DNA fragments that differ at the end position of the mutation specified by the primer.

The DNA fragments produced bearing the desired mutation can be used to replace the corresponding region in the plasmid that served as PCR template using standard DNA technology. Mutations at separate positions can be introduced simultaneously by either using a mutant second primer or performing a second PCR with different mutant primers and ligating the two resulting PCR fragments simultaneously to the vector fragment in a three (or more) part ligation.

Another method for preparing variants, cassette mutagenesis, is based on the technique described by Wells et al. Gene 34, 315, 1985. The starting material can be the plasmid (or vector) comprising the SOCS-3 DNA to be mutated. The codon(s) within SOCS-3 or SOCS-1 to be mutated are identified. Typically unique restriction endonuclease sites are present on each side of the identified mutation site(s). If such restriction sites do not exist, they can be generated using the above-described oligonucleotide-mediated mutagenesis method to introduce them at appropriate locations in the SOCS-3 or SOCS-1 DNA. After the restriction sites have been introduced into the plasmid, the plasmid is cut at these sites to linearize it. A double stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ ends that are compatible with the ends of the linearized plasmid, such that it can be directly ligated to the plasmid. The plasmid now contains the mutated SOCS-3 or SOCS-1 DNA sequence, that can be expressed to produce SOCS-3 or SOCS-1 with altered activity.

Further, SOCS-3 or SOCS-1 inhibitors/antagonists can function at the genetic level. Such antagonists include agents which decrease, inhibit, block or abrogate SOCS-3 or SOCS-1 expression, production or activity. Such an agent can be an antisense nucleic acid or sequence specific peptide nucleic acid. In addition, such an antagonist may interfere with SOCS-3 or SOCS-1 promoter activity. Further, such an antagonist can be a SOCS-3 or SOCS-1 mutant such as a mutant that functions as a competitive inhibitor which can be introduced and expressed in the cell where SOCS-3 activity is to be reduced. The mutant can be a full length derivative of SOCS-3 or SOCS-1 or fragments or derivatives as described above, such that expression of the mutant in a cell, inhibits the endogenous SOCS-3 or SOCS-1 activity. Such antagonists can be introduced into a cell by transfection, for example calcium phosphate precipitation, lipofection or by infection with a virus or pseudovirus containing the desired construct, by electroproration or by gene gun. The nucleic acids of the present invention can also be taken up by cells wherein the nucleic acid comprises naked DNA. The nucleic acid can be linear or in plasmid or circular form. Methods of introducing nucleic acid into a cell and tissues are well known in the art.

SOCS expression can be inhibited or reduced by introducing into a cell with a polynucleotide encoding SOCS antisense nucleic acid. Methods of inhibiting expression using antisense inhibition of expression are well known in the art, see for example Iversen et al., “Anti-Cancer Drug Design”, Vol. 6 pp. 531-538 and “In Vivo Studies With Phosphorothioate Oligonucleotides: Pharmacokinetic Prologue”; Iverson P. L. published by McMillan Press. 1991, 6531-6538. For example, the antisense molecule can hybridize to the endogenous SOCS mRNA such as SOCS-3 or SOCS-1 and prevent translation of said mRNA, thereby inhibiting or reducing expression of said SOCS protein. SOCS expression can also be inhibited or reduced by transfecting the cell with a polynucleotide construct encoding a transcriptional inhibitor such that transcription of SOCS protein such as SOCS-3 or SOCS-1 is inhibited or reduced. Such a transcriptional inhibitor interacts, for example, specifically with SOCS-3 or SOCS-1 promoter sequences, resulting in decreased transcription of said SOCS protein, decreased SOCS protein expression and thus decreased SOCS activity.

Nucleic acid as used herein refers to DNA, RNA or peptide nucleic acid (PNA). As used herein, the term “Peptide Nucleic Acid” or “PNA” includes compounds referred to as Peptide Nucleic Acids in U.S. Pat. Nos. 5,539,082, 5,527,675, 5,623,049 or 5,714,331 (herein incorporated by reference). Further modifications of PNA are well known in the art. Furthermore, like DNA, the backbone of the PNA can be modified, for example, to comprise phosphono-PNA.

In another embodiment, the present invention encompasses introducing into a cell a nucleotide expression construct into cells, wherein said construct encodes a modified form of SOCS-3 or SOCS-1. A modified form of SOCS-3 or SOCS-1 can include a dominant negative SOCS-3 or SOCS-1. Such a molecule can competitively bind the SOCS-3 or SOCS-1 target molecule without inactivating said target molecule (e.g., a dominant negative SOCS-3 or SOCS-1 would bind its target molecule, such as JAK2 and prevent the endogenous SOCS from binding, such that JAK2 remains phosphorylated, and/or such that JAK2 remains capable of phosphorlyating the appropriate downstream molecules, such as the cytokine receptor or STAT molecule.

Several vectors for use in such constructs are well known in the art. For example, recombinant expression vectors which include synthetic or cDNA-derived DNA fragments encoding modified SOCS-3 or SOCS-1 molecules comprising DNA encoding a modified SOCS-3 or SOCS-1 protein operably linked to suitable transcriptional or translational regulatory elements derived from mammalian, microbial, viral or insect genes. Such regulatory elements include a transcriptional promoter, an optional operator sequence to control transcription, a sequence encoding suitable mRNA ribosomal binding sites, and sequences which control the termination of transcription and translation, as described in detail below. The ability to replicate in a host, usually conferred by an origin of replication, and a selection gene to facilitate recognition of transformants may additionally be incorporated.

Operably linked indicates that components are linked in such a manner that expression of the DNA encoding a fusion protein is controlled by the regulatory elements. Generally, operably linked means contiguous.

Mammalian expression vectors may comprise non-transcribed elements such as an origin of replication, a suitable promoter and enhancer linked to the gene to be expressed, and other 5′ or 3′ flanking nontranscribed sequences, and 5′ to 3′ nontranslated sequences, such as necessary ribosome binding sites, a poly-adenylation site, splice donor and acceptor sites, and transcriptional termination sequences.

The transcriptional and translational control sequences in expression vectors to be used in transforming vertebrate cells may be provided by viral sources. For example, commonly used promoters and enhancers are derived from Polyoma, Adenovirus 2, Simian Virus 40 (SV40), and human cytomegalovirus. DNA sequence derived from the SV40 viral genome, for example, SV40 origin, early and late promoter, enhancer, splice, and polyadenylation sites may be used to provide the other genetic elements required for expression of a heterologous DNA sequence. The early and late promoters are particularly useful because both are obtained easily from the virus as a fragment which also contains the SV40 viral origin or replication. Smaller or larger SV40 fragments may also be used, provided the approximately 250 bp sequence extending from the Hind III site toward the BgII site located in the viral origin or replication is included. Exemplary vectors can be constructed as disclosed by Okayama and Berg (Mol Cell Biol 3:280, 1983. Preferred eukaryotic vectors for expression of mammalian DNA include pIXY321 and pIXY344, both of which are yeast expression vectors derived from pBC102.K22 (ATCC 67,255) and yeast.

The inhibitor compounds of the present invention include molecules that interact with endogenous SOCS-3 or SOCS-1 or to their target molecules such as JAK2, such that upon interacting with said molecules, inhibitor the SOCS mediated inhibition of CNTF cell-signaling activity. Encompassed by the present invention are inhibitor compounds that mimic the structure and conformation of the substrate moiety when interacting with the binding or active site. Molecular inhibitors of the present invention will typically have an inhibition constant (Kᵢ) of ten micromolar, or less. Specifically encompassed are organic molecules that mimic the structure and conformation of SH2 binding domains and interact with SOCS-3 or SOCS-1, thereby inhibiting their activity. In one embodiment, the inhibitor contains or mimics phosphotyrosine. Also encompassed by the present invention are small organic molecules that mimic the structure of SOCS-3 or SOCS-1, or, alternatively, the binding site of the SOCS-3 or SOCS-1 target, and therefore interfere with the interaction of SOCS-3 or SOCS-1 with its intended target molecule.

Peptides or organic molecules suitable for use as SOCS-3 or SOCS-1 inhibitors can be produced in libraries. The libraries of peptides or organic molecules comprise a mixture of substantially equimolar amounts of peptides or organic molecules. In one embodiment, the library can be designed to mimic SOCS-3 or SOCS-1 target molecules, e.g., JAK2. In another embodiment, the library comprises peptides or phosphotyrosine containing peptides or organic molecules that interact with the SH2 domain of SOCS-3 or SOCS-1, thereby inhibiting the ability of SOCS-3 or SOCS-1 to bind target molecules.

The inhibitors of the present invention can be synthesized using standard laboratory methods that are well known to those of skill in the art, including standard solid phase techniques. Inhibitors comprising naturally occurring amino acids can also be produced by recombinant DNA techniques known to those of ordinary skill in the art, and subsequently phosphorylated.

The inhibitors of the present invention can comprise either the 20 naturally occurring amino acids or other synthetic amino acids. Synthetic amino acids encompassed by the present invention include, for example, naphthylalanine, L-hydroxypropylglycine, L-3,4-dihydroxyphenylalanyl, α-amino acids such as L-α-hydroxylysyl and D-α-methylalanyl, L-α-methyl-alanyl, β amino-acids such as β-analine, and isoquinolyl.

D-amino acids and other non-naturally occurring synthetic amino acids can also be incorporated into the inhibitors of the present invention. Incorporation of D-amino acids and other non-natural amino acids is expected to prolong the half life of the inhibitors of the present invention by rendering them resistant or less susceptible to, e.g., proteases. Such other non-naturally occurring synthetic amino acids include those where the naturally occurring side chains of the 20 genetically encoded amino acids (or any L or D amino acid) are replaced with other side chains of the 20 genetically encoded amino acids (or any L or D amino acid) are replaced with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclic. In particular, proline analogs in which the ring size of the proline residue is changed from 5 members to 4, 6, or 7 member can be employed. As used herein, “lower alkyl” refers to straight and branched chain alkyl groups having from 1 to 6 carbon atoms, such as methyl, ethyl propyl, butyl and so on. “Lower alkoxy” encompasses straight and branched chain alkoxy groups having from 1 to 6 carbon atoms, such as methoxy, ethoxy and so on.

Cyclic groups can be saturated or unsaturated, and if unsaturated, can be aromatic or non-aromatic. Heterocyclic groups typically contain one or more nitrogen, oxygen, and/or sulphur heteroatoms, e.g., furazanyl, furyl, imidazolidinyl, imidazolyl, imidazolinyl, isothiazolyl, isoxazolyl, morpholinyl (e.g. morpholino), oxazolyl, piperazinyl (e.g., 1-piperazinyl, pyridyl, pyrimidinyl, pyrrolidinyl (e.g. 1-pyrrolidinyl), pyrrolinyl, pyrrolyl, thiadiazolyl, thiazolyl, thienyl, thiomorpholinyl (e.g. thiomorpholino), and triazolyl. The heterocyclic groups can be substituted or unsubstituted. Where a group is substituted, the substituent can be alkyl, alkoxy, halogen, oxygen, or substituted or unsubstituted phenyl. (See U.S. Pat. Nos. 5,654,276 and 5,643,873, the teachings of which are herein incorporated by reference).

Peptide mimetics that mimic the SOCS-3 or SOCS-1 protein can also be designed to inhibit SOCS-3 or SOCS-1 activity, thereby resulting in an increase of CNTF activity. These mimetics can be designed and produced by techniques known to those of skill in the art. (See e.g., U.S. Pat. Nos. 4,612,132; 5,643,873 and 5,654,276, the teachings of which are herein incorporated by reference). These mimetics are based on the SOCS-3 or SOCS-1 sequence, and possess activity antagonistic to the biological activity of the corresponding peptide compound, but possess a “biological advantage” over the corresponding peptide inhibitor with respect to one, or more, of the following properties: solubility, stability, and susceptibility to hydrolysis and proteolysis.

Methods for preparing peptide mimetics include modifying the N-terminal amino group, the C-terminal carboxyl group, and/or changing one or more of the amino linkages in the peptide to a non-amino linkage. Two or more such modifications can be coupled in one peptide mimetic inhibitor. Examples of modifications of peptides to produce peptide mimetics are described in U.S. Pat. Nos. 5,643,873 and 5,654,276, the teachings of which are incorporated herein by reference. Peptide mimetic libraries can also be produced as described above.

Alternatively, the SOCS-3 or SOCS-1 inhibitor can be an antibody or antibody fragment that interacts with SOCS-3 or SOCS-1, thereby preventing SOCS-3 or SOCS-1 from interacting with downstream target molecules such as JAK2, or such that SOCS-3 or SOCS-1 interact with JAK2 without interfering with JAK2 kinase activity. The term “antibody” is meant to encompass polyclonal antibodies, monoclonal antibodies (mAbs), chimeric antibodies (e.g., humanized antibodies) and antibody fragments that retain the biological activity of specific binding to SOCS-3 or SOCS-1, such as Fab, Fab′, F(ab′)2 and Fv. Also encompassed are single-chain antibodies (sFvs). These antibody fragments lack the Fc portion of an intact antibody, clear more rapidly from the circulation and can have less non-specific tissue binding than an intact antibody. These fragments are produced by well-known methods in the art, for example by proteolytic cleavage with enzymes such as papain (to produce Fab fragments) or pepsin (to produce F(ab′)2 fragments.

Polyclonal antibodies are heterogeneous populations of antibody molecules derived from the sera of animals immunized with an antigen. A monoclonal antibody (mAb) contains a substantially homogenous population of antibodies specific to antigens, which population contains substantially similar epitope binding sites. MAbs may be obtained by methods known to those skilled in the art. See, for example Kohler and Milstein, Nature 256:495-497, 1975; U.S. Pat. No. 4,376,110; Ausubel et al, eds., Current Protocols in Molecular Biology, Green Publishing Assoc. and Wiley Interscience, N.Y., 1987, 1992; and Harlow and Lane Antibodies: A Laboratory Manual Cold Spring Harbor Laboratory, 1988; Colligan et al., eds., Current Protocols in Immunology, Greene Publishing Assoc. and Wiley Interscience, N.Y., 1992, 1993; the contents of which references are incorporated entirely herein by reference. Such antibodies can be of any immunoglobulin class including IgG, IgM, IgE, IgA, and any subclass thereof. A hybridoma producing a mAb of the present invention can be cultivated in vitro, in situ, or in vivo. Production of high titers of mAbs in vivo or in situ makes this the presently preferred method of production.

Chimeric antibodies which include humanized antibodies, are molecules wherein different portions of which are derived from different animal species, such as those having variable regions derived from a murine mAb and a human immunoglobulin constant region. Chimeric antibodies are primarily used to reduce immunogenicity in application and/or to increase yields in production, for example. Chimeric antibodies and methods for their production are known in the art.

Typically, antibodies of the present invention are high affinity anti-SOCS-3 or SOCS-1 antibodies, and fragments or regions thereof, that have potent inhibiting and/or neutralizing activity in vivo against SOCS-3 or SOCS-1. Such antibodies can include those generated by immunization using purified recombinant SOCS-3 or SOCS-1 or peptide fragments thereof. Methods for determining antibody specificity and affinity can be found in Harlow, et al., Antibodies: A Laboratory Manual, Cold spring Harbor Laboratory Press, Cold Spring Harbor, N. Y., 1988; Colligan et al., eds., Current Protocols in Immunology, Greene Publishing Assoc. and Wiley Interscience, N.Y., 1992, 1993; and Muller, Meth. Enzymol., 92:589-601 1983; which references are entirely incorporated herein by reference.

The antagonists/agonists of the present invention can be formulated into compositions with an effective amount of the inhibitor/antagonist/agonist as the active ingredient. An effective amount of a SOCS-3 or SOCS-1 inhibitor/antagonist is an amount effective to partially or completely inhibit SOCS-3 or SOCS-1 activity resulting in increased CNTF activity. An effective amount of a SOCS-3 or SOCS-1 agonist is an amount effective to enhance SOCS-3 or SOCS-1 activity resulting in a decrease of CNTF activity. Methods to evaluate CNTF activity, such as monitoring food intake, energy expenditure, blood glucose and weight gain/loss are well-known to those of skill in the art. It will be appreciated that the actual effective amounts of the inhibitor/antagonist/agonist in a specific case will vary according to the specific compound being utilized, the particular composition formulated, the mode of administration and the age, weight and condition of the mammal, for example. Dosages for a particular mammal can be determined by one of ordinary skill in the art using conventional considerations, (e.g. by means of an appropriate, conventional pharmacological protocol).

Such compositions can also comprise a pharmaceutically or physiologically acceptable carrier, and are referred to herein as pharmaceutical compositions. The compositions of the present invention can be administered intravenously, parenterally, orally, by transdermal patch, by inhalation or by suppository. The inhibitor/antagonist/agonist composition may be administered in a single dose or in more than one dose over a period of time to achieve a level of inhibitor/antagonist/agonist which is sufficient to confer the desired effect.

Suitable pharmaceutical or physiological carriers include, but are not limited to water, salt solutions, alcohols, polyethylene glycols, gelatin, carbohydrates such as lactose, amylose or starch, magnesium stearate, talc, silicic acid, viscous paraffin, fatty acid esters, hydroxymethylcellulose, polyvinyl pyrolidone, etc. The pharmaceutical preparations can be sterilized and desired, mixed with auxiliary agents, e.g., lubricants, preservatives, stabilizers, wetting agents, emulsifiers, salts for influencing osmotic pressure, buffers, coloring, and/or aromatic substances and the like which do not deleteriously react with the active compounds. They can also be combined where desired with other active agents, e.g., enzyme inhibitors, to reduce metabolic degradation.

For parenteral application, particularly suitable are injectable, sterile solutions, preferably oily or aqueous solutions, as well as suspensions, emulsions, or implants, including suppositories. Ampules are convenient unit dosages.

The inhibitors/antagonists/agonists of the present invention can be administered to an individual mammal in need of such treatment, in conjunction with an agent or agents that allow the inhibitor to pass through the blood brain barrier. The inhibitor/antagonist/agonist and the agent can be administered simultaneously or sequentially. Such agents are known in the art, such as those described in U.S. Pat. Nos. 5,112,596; 5,268,164; 5,686,416 and 5,506,206; the teachings of which are incorporated herein by reference in their entirety.

The following Examples are offered for the purpose of illustrating the present invention and are not to be construed to limit the scope of this invention.

### EXAMPLES

### Example 1

Activation of SOCS-3 mRNA by CNTF and leptin in hypothalamus from ob/ob mice.

The following primers were used for specific PCR amplification of cis, SOCS-1, SOCS-2 and SOCS-3 cDNAs:

CIS-1A: 5′-ctggagctgcccgggccagcc-3′, SEQ ID NO:1 and CIS-1B:

5′-caaggctgaccacatctggg-3′, SEQ ID NO:2 (producing 400 bp product (GenBank Acc. Number D31943);

SOCS-1A: 5′-ccactccgattaccggcgcatc-3′, SEQ ID NO:3 and SOCS-1B:

5′-gctcctgcagcggccgcacg-3′, SEQ ID NO:4 (producing 350 bp product (GenBank Accession Number U88325);

SOCS-2A: 5′-aagacgtcagctggaccgac-3′, SEQ ID NO:5 and

SOCS-2B: 5′-tcttgttggtaaaggcagtccc-3′, SEQ ID NO:6 (producing 300 bp product (GenBank Acc. Number U588327);

SOCS-3A: 5′-accagcgccacttcttcacg-3′, SEQ ID NO:7 and

SOCS-3B: 5′-gtggagcatatactgatcc-3′, SEQ ID NO:8 (producing 450 bp product (GenBank Acc. Number U88328).

Each 50 μl PCR reaction was carried out with 5.0 μl of cDNA as template. The assay conditions were: 10 mM Tris-HCl (pH 8.8), 50 mM KCl, 1.5 mM MgCl,. 0.01% gelatin 0.2 mM dNTPs. 20 pmol of each primer as described above. 2.5 units of Taq polymerase (Stratagene) and 1.0 μl of α³²P-dCTP (29.6 TBq/mmol. 370 MBq/ml)(NEN, Boston, Mass.). The mixture was overlaid with 25 μl of mineral oil, and after initial denaturation at 96° C. for 3 min, the samples were subjected to 25 cycles of amplification: denaturation at 95° C. for 1 min, annealing at 60° C. for 1 min, and extension at 72° C. for 45 seconds. Ten μl of the reaction were then combined with 5 μg of sequencing stop solution (Amersham International, Buckinghamshire, UK) and heated to 85° C. for five minutes before loading 5 μl onto a 4% urea-acrylamide gel (38×31×0.03 cm). Electrophoresis was carried out at 60 W of constant power four hours, before the gels were transferred to filter paper, dried and finally subjected to ³²P quantification by PhosphorImager™ analysis (Molecular Dynamics).

Preliminary PCR experiments showed that the rate of amplification was linear for CIS-1, SOCS-1 and SOCS-3 when applying less than 30 PCR-cycles. The amplification rate of SOCS-2 was linear for 27 cycles, after which non-linear amplification appeared. Twenty five cycles of PCR amplification were used for quantification of CIS-1, SOCS-1, SOCS-2 and SOCS-3. PCR reactions were spiked with ³²P-dCTP and assembled in parallel for each cDNA and subjected to PCR amplification under the above conditions of limiting number of cycles. PCR products were then separated on denaturing acrylamide gels and finally subjected to autoradiography.

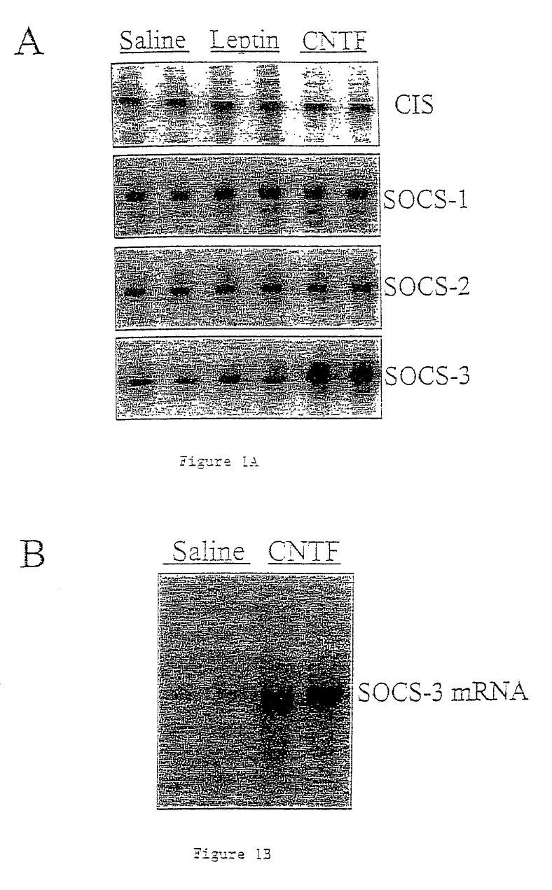

Ad libitum fed male ob/ob mice (Jackson Laboratories, Bar Harbor, Me.) aged 7-8 weeks were injected intraperitoneally (ip) with 10 μg of recombinant human CNTF (Eli Lilly, Indianapolis, Ind.), or 100 μg recombinant mouse leptin (Eli Lilly), or saline. Two hours later, total RNA was purified from hypothalami, and quantitative ³²P-RT-PCR for CIS, SOCS-1, SOCS-2 and SOCS-3 mRNA's was performed. CNTF treatment resulted in robust increase of SOCS-3 mRNA in the hypothalamus, as shown in FIG. 1A. This was confirmed by Northern blot analysis (FIG. 1B). No effects on CIS, SOCS-1 or SOCS-2 mRNA were detected in this tissue (FIG. 1A). Leptin treatment caused a modest increase of SOCS-3 mRNA, but not of CIS, SOCS-1, or SOCS-2 mRNA (FIG. 1A). PhosphorImager™ analysis demonstrated a 6-15 fold and a 2 fold increase in SOCS-3 mRNA after CNTF and leptin treatment, respectively. Similar effects on SOCS-3 mRNA were seen 1 or 3 hours after treatment.

### Example 2

SOCS-3 mRNA is differentially activated by CNTF and leptin in the hypothalamus.

In order to localize the specific anatomic regions of the hypothalamus and other parts of the brain in which leptin affects SOCS-3 mRNA levels, ³⁵S-labeled RNA antisense probe was generated. The SOCS-3 A and SOCS-3B primers from above were used amplify a 450 base pair fragment of the mouse SOCS-3 cDNA. The PCR products were cloned into pCR2.1 (Invitrogen, Carlsbad, Calif.) according to the manufactures recommendations. The orientation of the cloned cDNA was verified by sequencing using standard double-stranded plasmid techniques. For generation of sense ³⁵S-labeled RNA, the plasmid was linearized by digestion with BamHI, and subjected to in vitro transcription with T7 polymerase according to the manufactures protocols (Promega). In situ hybridization histochemistry was conducted according to methods well known in the art (Simmons). Tissue sections of mouse and rat brain were mounted onto slides, air dried, and stored in desiccated boxes at −20° C. Prior to hybridization, the slides were immersed in 10% neutral buffered formalin, incubated in 0.001% proteinase K (Boehringer Mannheim) for 30 min., then in 0.025% acetic anhydride for 10 min., and dehydrated in ascending concentrations ethanol. The RNA probes were then diluted to 10⁶ cpm/ml in hybridization solution of 50% formamide, 10 mM Tris-HCl, pH 8.0, 5 mg tRNA, 10 mM dithiothreitol, 10% dextran sulfate, 0.3 M NaCl, 1 mM EDTA, pH 8, and 1× Denhardt's solution (Sigma). Hybridization solution and a glass coverslip was applied to each slide and sections were then incubated for 12-16 hours at 56° C. The coverslips were removed and the slides washed 4 times with 4× SSC. Sections were then incubated in 0.002% RNAase A (Boehringer Mannheim) with 0.5 M NaCl, 10 mM Tris-HCl, pH 8, and 1 mM EDTA, for 30 min. at 37° C. Sections were rinsed in decreasing concentrations of SSC containing 0.25% DTT: 2× at 50° C. for 1 hour, 0.2× at 55° C. for 1 hour, and 0.2× for 1 hour at 60° C. Sections were next dehydrated in graded ethanol (50, 70, 80, and 90%) containing 0.3 M NH₄OAc followed by 100% ethanol. Slides were air dried and placed in X-ray film cassettes with BMR-2 film (Kodak) for 3-5 days. Slides were then dipped in NTB2 photographic emulsion (Kodak), dried and stored with desiccant in foil-wrapped slide boxes at 4° C. for 2-3 weeks. Slides were developed with D-19 developer (Kodak), counterstained with thionin, dehydrated in graded ethanol, cleared in xylene, and coverslipped with Permaslip. Sections were analyzed with a Zeiss Axioplan light microscope using brightfield and darkfield optics. Photomicrographs were produced by capturing images with a digital camera (Kodak, DCS) mounted directly on the microscope and an Apple Macintosh Power PC computer. Image editing software (Adobe Photoshop) was used to combine photomicrographs into plates and figures were printed on a dye sublimation printer (Kodak 8600). Only the sharpness, contrast, and brightness were adjusted.

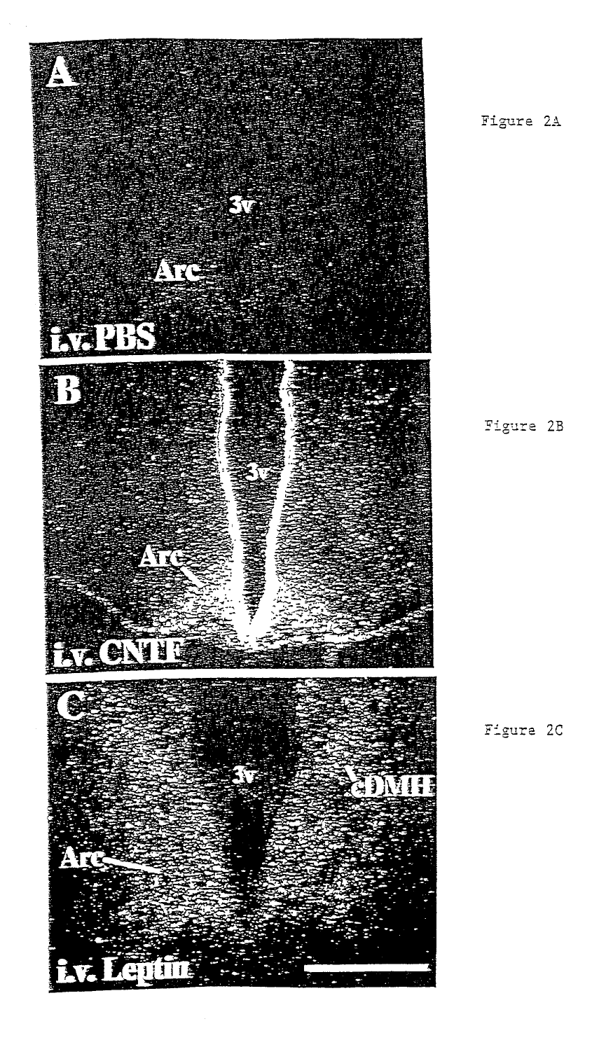

The results are shown in FIG. 2. Regions of the hypothalamus in which CNTF and leptin induce SOCS-3 mRNA were identified and compared by in situ hybridization histochemistry. In normal rats given a single intravenous (iv) injection of recombinant human CNTF (0.25 μg/g body weight), a marked increase in specific hybridization was detected in the arcuate nucleus as compared to saline injected rats (FIGS. 2A, 2B). A significant proportion of the SOCS-3 expressing cells in this region were of neuronal nature as determined by the characteristic clustering of silver grains. The ependymal lining of all ventricles, the median eminence and the subfornical organ also exhibited high intensity specific signals. Similar results were obtained in ob/ob mice.

The SOCS-3 hybridization pattern obtained after CNTF treatment was different from that obtained after leptin treatment (FIG. 2C). After intravenous injection of murine leptin (1 μg/g body weight), a marked increase in specific hybridization was detected in the arcuate nucleus (Arc) and the dorsomedial hypothalamic nucleus (DMH). No specific signals were detected in the subfornical organ or in the ependymal lining of the ventricles. The Arc and the DMH are those regions of the hypothalamus in which the long form of the leptin receptor is most highly expressed. The hybridization pattern obtained in the Arc with CNTF overlaps with those regions expressing neuropeptide Y (NPY), pro-opiomelanocortin (POMC), cocaine- and amphetamine-regulated transcript (CART) and agouti-related-protein (AGRP), all of which are peptides involved in regulation of feeding that are regulated by leptin in vivo.

### Example 3

CNTF activates SOCS-3 mRNA and SOCS-3 protein in astrocytes and forced expression of SOCS-3 inhibits CNTF-induced signal transduction.

As demonstrated herein, CNTF directly induces SOCS-3 gene expression. A CNTF responsive human astrocyte cell line was used to measure activation of the JAK-STAT pathway using an electrophoretic-mobility shift assay specific for activated STAT1 and STAT3 (Schreiber, E., et al., Nucleic Acids Res., 17:6419 (1989);Wagner, B. J., et al., EMBO J., 9:4477-4484 (1990)). Astrocytes were grown to confluence in 6 well dishes and serum-deprived 12-15 hours prior to stimulation with hormones. After treatment, cells were rinsed once with 2 ml of ice-cold Tris-buffered-saline (TBS) and then scraped into 1.0 ml of ice-cold TBS, transferred to a 1.5 ml eppendorf tube and pelleted by centrifugation at 1500× g at 4° C. for 5 minutes. The pellets were then resuspended in 400 μl ice-cold buffer C (40 mM HEPES pH 7.9, 10 mM KCl, 0.1 mM EDTA, 0.1 mM EGTA, 1 mM DTT, 0.5 mM PMSF) by gentle pipetting in a yellow tip. The cells were allowed to swell on ice for 15 min., after which 25 μl of 10% Nonidet NP-40 were added and the tube vortexed for 10 seconds. Samples were then centrifuged for 30 sec at 14,000× g and the nuclear pellets resuspended in 25 μl of ice-cold buffer D (20 mM HEPES pH 7.9, 0.4 M NaCl, 1 mM EDTA, 1 mM EGTA, 1 mM DTT, 1 mM PMSF) by vigorous rocking at 4° C. for 30 minutes. The nuclear extracts were finally clarified by centrifugation at 14,000× g for 20 minutes and stored at −80° C. until further use. Five μg of nuclear extracts (determined by Bradford protein assay, Bio-Rad, Hercules, Calif.) were added to binding buffer (final volume was 20 μl: 13 mM HEPES pH 7.9, 65 mM NaCl, 1 mM DTT, 0.15 mM EDTA, 8% glycerol, 50 mg/ml poly (dI-dC), and 0.01% NP-40) which included 100,000 cpm of the ³²P-labeled double-stranded oligonucleotide probe (SIE-mutant 67 (Wagner, B. J., et al., EMBO J., 9:4477-4484 (1990)), and incubated for 15 min at room temperature. The probe was generated by annealing two oligonucleotides: 5′-CGCTCCATTTCCCGTAAATCAT-3′ (SEQ ID NO: 9) and 5′-CGCTCATGATTTACGGGAAATG-3′ (SEQ ID NO: 10), followed by a fill-in reaction of the 5 base overhangs using T7 polymerase (GIBCO-BRL) and ³²P-α-dNTP's (each 222 TBq/mmol, 740 MBq/ml)(NEN, Boston, Mass.). Unincorporated nucleotides were removed by using a G25 Quick Spin column (Boehringer Mannheim, Indianapolis, Ind.). Samples were loaded onto a 5% non-denaturing poly-acrylamide gel (39:1 acrylamide:bis) containing 2.5% glycerol in 0.5× Tris-Borate-EDTA (TBE) buffer and run for 1.5 hours at 220 V at 4° C. After drying, gels were placed in a PhosphorImager™ cassette (Molecular Dynamics, Sunnyville, Calif.) for 12-15 hours.

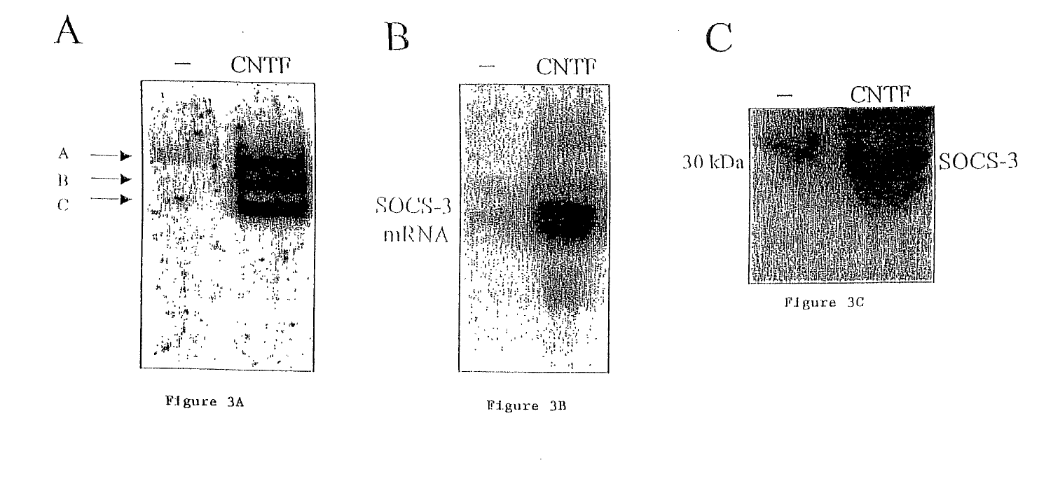

As shown in FIG. 3A, CNTF induced robust DNA binding activities of STAT1and STAT3 hetero- and homodimers. As determined by Northern blot analysis of RNA isolated from astrocytes, CNTF treatment resulted in strong induction of SOCS-3 mRNA (FIG. 3B). Furthermore, CNTF treatment of these cells generated a robust increase in cellular SOCS-3 protein levels as determined by Western blotting of SOCS-3 immunoprecipitates (FIG. 3C). Leptin did not activate STAT DNA binding activity or induce SOCS-3 mRNA in these cells.

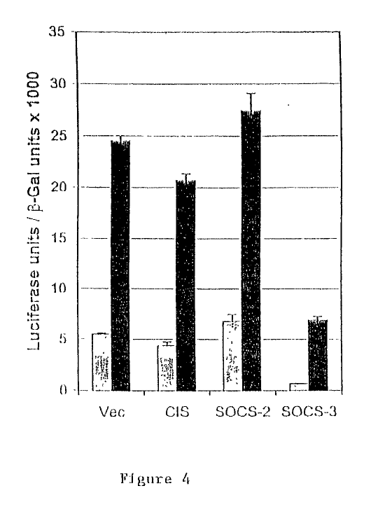

The effects of SOCS-3 on CNTF signaling were determined by measuring the effect of SOCS-3 on CNTF-induced transcription in astrocytes. Cells were grown in 6 well plates and transfected with a total of 2.0 μg of plasmid DNA (γ-interferon activated sequence-luciferase reporter construct, GAS-1uc) using 15 μl of Lipofectamine per well. Forty-eight hours post transfection, cells were lysed in 500 μl of 25 mM glycylglycine, 15 mM MgSO₄, 4 mM EGTA with 1% Triton X-100 and 2 mM DTT (lysis buffer A). After lysis, 50 μl aliquots were used for the luciferase assay. Briefly, 150 μl 0.75 mM luciferin (Molecular Probes, Eugene, Oreg.) and 150 μl assay buffer (lysis buffer A+15 mM K₂HPO₄, 6 mM ATP, 3 mM DTT, pH 7.6) were injected simultaneously and measured for 20 seconds by a Luminometer (LB 9501, EG&G Berthold, Bad Wildbad, Germany). β-galactosidase activities were determined in 20 μl samples using Galacton (Tropix Inc., Bedford, Mass.) as described by the manufacturer and measured for 5 seconds by the Luminometer. As shown in FIG. 4, SOCS-3, but not CIS or SOCS-2, inhibited CNTF-induced activation of a STAT-responsive luciferase reporter construct in transfected astrocytes.

### Example 4

CNTF activates multiple SOCS and CIS isoforms in cerebellum, kidney and liver.

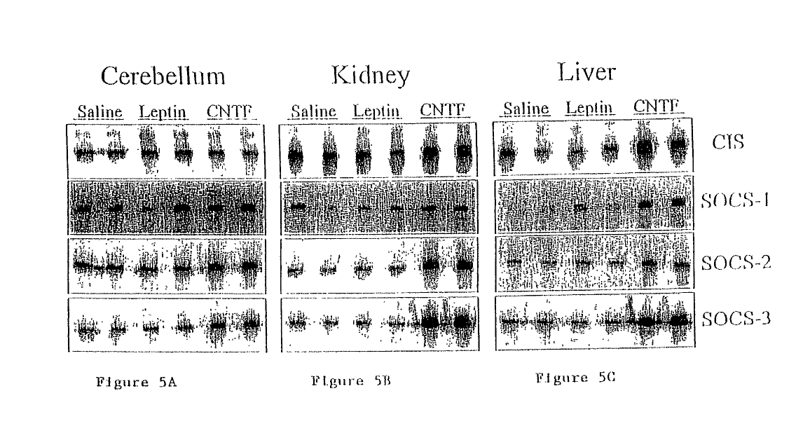

Peripherally administered CNTF acts on other regions of the brain and on peripheral tissues. In the cerebellum, CNTF specifically induced SOCS-3 mRNA as determined by quantitative RT-PCR (FIG. 5A), but no effects on CIS, SOCS-1 or SOCS-2 mRNA's were detected. In kidney, CNTF had a strong effect on both SOCS-2 and SOCS-3 mRNA levels (FIG. 5B). In the liver, however, all the tested genes, including cis, socs-1, socs-2 and socs-3 were induced after CNTF treatment (FIG. 5C). Leptin did not affect CIS or SOCS mRNA levels in cerebellum, kidney or in liver, suggesting that these tissues do not express significant levels of the long form of the leptin receptor.

Equivalents

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize or be able to ascertain using no more than routine experimentation, many equivalents to the specific embodiments of the invention described specifically herein. Such equivalents are intended to be encompassed in the scope of the claims.

What is claimed is:

1. A method of modulating ciliary neurotrophic factor cell signaling activity in a cell, comprising contacting said cells with an modulator of SOCS-3 activity.

2. The method of claim 1 wherein SOCS-3 activity is inhibited, resulting in increased ciliary neurotrophic factor cell signaling activity.

3. The method of claim 2 wherein the expression of SOCS-3 protein is inhibited, comprising introducing a nucleotide construct comprising a polynucleotide wherein the polynucleotide prevents transcription of SOCS-3 DNA.

4. The method of claim 2 wherein the expression of SOCS-3 protein is inhibited, introducing a nucleotide construct comprising a polynucleotide encoding SOCS-3 antisense nucleotide into a cell, wherein the antisense SOCS-3 nucleotide acid binds to endogenous SOCS-3 mRNA in the cell, thereby inhibiting expression of SOCS-3 protein.

5. The method of claim 2, comprising introducing a nucleotide construct encoding a modified SOCS-3 polypeptide into the cell, wherein the modified SOCS-3 polypeptide is a competitive inhibitor of endogenous SOCS-3, thereby inhibiting SOCS-3 activity.

6. The method of claim 2, comprising introducing a SOCS-3 inhibitor into a cell, wherein the inhibitor interferes with the interaction of SOCS-3 with a SOCS-3 target protein.

7. The method of claim 6, wherein the SOCS-3 target protein is JAK2.

8. The method of claim 1, wherein SOCS-3 activity is increased, resulting in decreased ciliary neurotrophic factor cell signaling activity.

9. The method of claim 8, wherein the expression of SOCS-3 protein is increased.

10. The method of claim 9, comprising introducing into a cell a nucleotide construct encoding a SOCS-3 polypeptide or a modified SOCS-3 polypeptide.

11. A cell line comprising a cytokine receptor and a reporter gene construct, wherein contacting said cytokine receptor with its cognate ligand results in SOCS-3 production, and wherein transcription of the reporter gene is inhibited by SOCS-3.

12. The cell line of claim 11, wherein the reporter gene construct contains SOCS-3 promoter elements.

13. The cell line of claim 11, wherein the cytokine receptor is the ciliary neurotrophic factor receptor.

14. A method for identifying inhibitors of SOCS-3 activity, comprising the steps of:
a) contacting the cells of claim 11 with an organic molecule library comprising candidate SOCS-3 inhibitors or transfecting said cells with a cDNA expression library comprising DNA encoding candidate SOCS-3 inhibitors;
b) contacting the cells of step a) with ciliary neurotrophic factor;
c) selecting the cells of step b) having increased reporter gene activity; and
d) identifying the organic molecule or cDNA selected in step c).

15. A SOCS-3 inhibitor identified by the method of claim 14.

16. A ciliary neurotrophic factor responsive cell line, wherein the cell line is dependent upon a second cytokine for growth.

17. The cell line of claim 16, wherein the second cytokine is IL-3.

18. A method for identifying inhibitors of SOCS-3 activity comprising the steps of:
a) culturing the cells of claim 17 in the presence of IL-3 under conditions suitable for growth;
b) removing the cells of step a) from the presence of IL-3;
c) contacting the cells of step b) with an organic molecule library comprising candidate SOCS-3 inhibitors or transfecting said cells with a cDNA expression library comprising candidate SOCS-3 inhibitors;
d) contacting the cells of step c) with ciliary neurotrophic factor;
e) selecting the cells of d), that are capable of proliferating in the presence of ciliary neurotrophic factor; and
f) identifying the organic molecule or cDNA selected in e).

19. A SOCS-3 inhibitor identified by the method of claim 18.

20. A method of reducing weight or food intake in a mammal, comprising administering an effective amount of a SOCS-3 inhibitor to said mammal.

21. A method of reducing weight or food intake in a mammal, comprising administering an effective amount of ciliary-neurotrophic factor in combination with a SOCS-3 inhibitor to said mammal.

22. A method of preventing or inhibiting neurodegeneration in a mammal, comprising administering an effective amount of a SOCS-3 inhibitor to said mammal.

23. A method of preventing or inhibiting neurodegeneration in a mammal, comprising administering an effective amount of ciliary neurotrophic factor in combination with a SOCS-3 inhibitor to said mammal.

24. A method of increasing weight or food intake in a mammal, comprising administering an effective amount of ciliary neurotrophic factor inhibitor to said mammal.

25. The method of claim 24, wherein SOCS-3 activity is enhanced.
